(19) United States
(12) Patent Application Publication
(10) Pub. No.: US 2024/0282407 A1
(43) Pub. Date: Aug. 22, 2024

(54) ELECTRONIC VARIANT CLASSIFICATION

(71) Applicant: Myriad Genetics, Inc., Salt Lake City, UT (US)

(72) Inventors: Dmitry Pruss, Salt Lake City, UT (US); Brian Morris, Salt Lake City, UT (US); Karla Bowles, Salt Lake City, UT (US); Julie M. Eggington, Salt Lake City, UT (US); Benjamin B. Roa, Salt Lake City, UT (US); Alexander Gutin, Salt Lake City, UT (US); Elisha Hughes, Salt Lake City, UT (US)

(73) Assignee: Myriad Genetics, Inc., Salt Lake City, UT (US)

(21) Appl. No.: 18/646,644

(22) Filed: Apr. 25, 2024

Related U.S. Application Data
(63) Continuation of application No. 14/209,703, filed on Mar. 13, 2014.
(60) Provisional application No. 61/799,813, filed on Mar. 15, 2013.

(51) Int. Cl. G16B 20/20; G16B 20/00

(52) U.S. Cl. CPC G16B 20/20; G16B 20/00

(57) ABSTRACT

A computer-implemented method is discussed that includes identifying, by a computer server system, stored electronic data that represents genetic sequencing for one or more genes for individuals in a population of patients who have submitted to genetic sequencing; generating, for each of multiple individuals and from the stored electronic data, probability data for the individuals and probability or weighting data, or both, for relatives of the individuals, the probability data representing likelihoods that a particular person corresponding to the probability data carries a deleterious mutation in a particular gene; and generating a score for a genetic variant, wherein the score is a function of probability or weighting data, or both, for the individuals and for relatives of the individuals, and the score represent a composite probability that a certain variant is a deleterious or benign variant.

### CROSS-REFERENCE TO RELATED PATENT APPLICATIONS

This application is a continuation of U.S. patent application Ser. No. 14/209,703, filed Mar. 13, 2014, which claims the benefit of U.S. Provisional Application Ser. No. 61/799,813, filed Mar. 15, 2013. The disclosure of the prior applications is considered part of (and is incorporated by reference in) the disclosure of this application.

### TECHNICAL FIELD

The present disclosure relates to classification of variants into categories that may be more informative than categories into which the variants would otherwise be classified, and for which the classification could be indeterminate and ambiguous.

### BACKGROUND

Computer systems commonly play a central role in classification operations. For example, a large number of data points may exist in a population, and a computer system may be used to bin members of the population into a defined number of tiers based on one or more parameter values for various parameters measured for members of the population.

As one example, members of a patient population (often referred to as “probands”) may voluntarily undergo genetic testing to identify their susceptibility to contracting various diseases, such as certain types of cancer. Such probands may be classified into groups for different types of cancer based on genetic variants identified for each particular proband. As one such example, in women, breast cancer is the most common cause of cancer-related death and the second most commonly diagnosed cancer. It is estimated that 7% of breast cancers and 10% of ovarian cancers result from single gene mutations (variants that are very uncommon, e.g., occurring on the order of less than 1% of the members of a population). The majority of such mutations have been identified as occurring in the BRCA1 and BRCA2 genes.

DNA sequencing and large rearrangement analysis can be conducted on probands, with resulting variants assigned within a 5-tier classification system. These classifications, from most deleterious to most benign, are: (1) “Deleterious,” which is highly likely pathogenic; (2) “Suspected Deleterious,” which is likely to be pathogenic; (3) “Variant of Uncertain Clinical Significance” (VUS), which is indeterminate; (4) “Genetic Variant, Favor Polymorphism,” which is likely not to be pathogenic; and (5) Genetic Variant, Polymorphism, which is almost certainly not pathogenic. Generally, a patient with a variant in the first two categories will be clinically managed as having a high cancer risk, and will thus receive increased testing and follow-up observations. Patients in the middle, VUS, category generally do not receive such heightened care, and are left relatively uncertain about their condition, which can create real anxiety.

Efforts may be made to reclassify variants in the middle tier so as to allay concerns in patients for such variants, or to push such variants into a tier for which follow-up care will generally be prescribed. For example, Easton et al. used regression analysis to estimate the probability of a proband carrying a deleterious mutation, given the proband's personal and family history, and combined such probabilities to obtain a likelihood that a mutation is deleterious.

### SUMMARY OF THE INVENTION

This document discusses systems and techniques by which a computer system may analyze data for a large number of probands, and families of probands, for the purpose of assigning a clinical classification to a variant. Assignment is based on statistical analysis of co-occurrence of certain types of cancers, and ages at which the cancers were diagnosed, with particular genetic variants, such as variants in the BRCA1 and BRCA2 genes. The process described here analyzes data regarding variants across a large patient population, and assigns statistical probabilities or weights for the particular patients and for their families regarding correlations between each particular variant and particular types of cancers or other genetically-affected diseases of interest. Probability and weighting tables may thus be constructed for the particular patients and their families and stored electronically in a database, and the probabilities for each variant may be used to compute a likelihood ratio for each patient, and then by extension, a score for each particular variant, where the score for each variant is a function of the likelihood ratios for all of the patients in combination.

Such scores may then be checked against those of composite controls (both deleterious and benign) to determine their legitimacy. Specifically, to use patient information to determine if a genetic variant carried by the patient is more likely to be benign or hazardous (deleterious), a system can attempt to compare the personal and family health history of the particular patient (as condensed in one score) to the scores of a large matching cohort of similar patients who either have only benign variants, or harbor deleterious variants, and who serve as benign or deleterious controls, respectively. Thus, each patient is compared with a matched benign cohort and a match deleterious cohort. Therefore, the entire set of patients carrying a genetic variant can be compared with “composite variant” cohorts, built by randomly drawing one control at a time from each of the matching benign or deleterious controls for each patient. For a simple example, assume a dataset with just two patients, Pete and Steve. Pete is matched to Alex and Bill who only have benign variants. Steve is matched to Mark and Jim who only have benign variants. So one of the randomly-drawn benign composites may be Alex and Mark, another Alex and Jim, and then maybe Bill and Jim, etc. In a typical implementation, there would be much more than two patients, and each of them will be matched to many more than two controls, so that there will be a very large variety of composites. But each composite has exactly as many subjects as the variant itself, and each of its subjects is stringently matched to a respective carrier of the variant.

Combinatorial random drawing from the matching control repeats, when repeated for each patient, yields a very large set of composite variants, each with its composite score. By comparing the distributions of benign and deleterious composite scores with the score for the actual variant, one may draw a statistically sound conclusion about the causal link between the variant and the disease (or lack thereof).

The use of matched control sets can also provide more effective information for a clinician. In the prior explanation, the attempt was to compare strengths of personal and family history of the patients, which is not invariant across all patient groups. Such a factor depends on access to health care, patient and physician awareness about genetic condition, and insurance coverage (all of which result in patients with less severe history of disease being tested). It depends on family size, family coherence, and lack of stigma associated with heritable conditions (all of which result in better knowledge, and/or stronger reporting, of the family history of disease). Ethnic and racial background, age, socio-economic status, and changing genetic health education and market penetration of the genetic tests, all ultimately affect the distribution of scores, and may be used for proper control matching. Ethnicity and time of testing are discussed below in one example for proper control matching, as these factors have been empirically demonstrated to exert the most significant effect on the observed range of severities of genetic conditions, and, hence, distribution of the scores. But other demographic and socio-economic factors may be employed as well to define matching controls appropriately, such as place of residence for custom analyses, when it also carried a strong impact on the scoring distribution (e.g. analyzing residents of Hawaii separately for a variant frequent in that state).

The probabilities determined via such a process may then be used to reclassify particular variants, either explicitly or implicitly. Explicit reclassification may involve, for example, moving a particular variant from classification as VUS, to either “Suspected Deleterious” (bad) or “Genetic Variant, Favor Polymorphism” (good). Implicit reclassification may occur by leaving the variant classified, technically and under a controlling standard, as VUS, but providing a physician or other caregiver with additional data, generated using the techniques described above, that allows the physician to treat the patient differently (e.g., more aggressively or less aggressively) than if the physician simply knew that the patient was VUS but did not have the extra data. Such action may have the same practical result as if the patient was explicitly reclassified. In particular, in a process that uses the techniques discussed here, an observation, testing, and treatment plan may be generated or modified to reflect the reclassification so that the particular patient (whose data may be rolled into a database with prior

In certain implementations, the techniques described here can provide for one or more advantages. For example, for certain genes, such as BRCA1 and BRCA2, segregation analysis may not be helpful because large pedigrees can be hard to obtain (and also, penetrance may be incomplete, and there may be a significant phenocopy rate). The techniques described here may provide statistical data that are not available from segregation analysis. The reclassification of variants as described here may also have a very low error rate, both negative and positive. Therefore, patients at the margin may receive more accurate assessments of their likelihood of contracting a disease and may be properly managed by their caregivers as a result.

In one implementation, a computer-implemented method comprises identifying, by a computer server system, stored electronic data that represents genetic sequencing for one or more genes for individuals in a population of patients who have submitted to genetic sequencing; generating, for each of multiple individuals and from the stored electronic data, probability data for the individuals and probability or weighting data, or both, for relatives of the individuals, the probability data representing likelihoods that a particular person corresponding to the probability data carries a deleterious mutation in a particular gene; and generating a score for a genetic variant, wherein the score is a function of probability or weighting data, or both, for the individuals and for relatives of the individuals, and the score represent a composite probability that a certain variant is a deleterious or benign variant. The probability information can be generated by identifying frequencies with which patients, other than a particular patient, who have a mutation that the particular patient has, are diagnosed with a relevant disease associated with a mutation in the relevant gene. Also, the patients can be categorized based on an age at which they were diagnosed with the relevant disease associated with the mutation, and the age at which the patients are determined to have been diagnosed with the relevant disease, can be adjusted based on a severity of the disease at the time of diagnosis.

In some aspects, the score for the genetic variant is a product of likelihoods for each of multiple patients identified as having the genetic variant, and also, the likelihoods for each of the multiple patients may represent a ratio of posterior odds over prior odds, and wherein the posterior odds represent a frequency with which patients with the genetic variant were determined to have been diagnosed with the disease associated with the genetic variant and the prior odds represent a frequency with which patients with the genetic variant were predicted to have a deleterious mutations, and the posterior odds represent actual rates of diagnosis for a disease associated with the mutation. Moreover, the probability data for the individuals and the relatives of the individuals can be generated for each gene of a plurality of different genes under investigation.

In yet other aspects, the probability data for relatives of the individuals is estimated for each relative by identifying carriers with corresponding types of cancer that were diagnosed at corresponding ages in relatives and corresponding carriers. The method may also comprise weighting the probabilities according to a distance of relationship between a particular patient and a relative of the patient who has been identified as having been diagnosed with a disease associated with the genetic variant. Moreover, the method may include comparing the score for the genetic variant to a corresponding score computed for a control populations having a corresponding mutation determined to be deleterious, and the method may also include comparing the score for the variant to a corresponding score computed for a control populations having a corresponding mutation determined to be benign. In other instances, the method can also include comparing the score for the variant to a corresponding score computed for a control populations having a corresponding mutation determined to be benign, and using the generated score to diagnose a patient having the genetic variant. The action of diagnosing the patient can comprise classifying or reclassifying the patient into one of a plurality of predefined classification bands for a variant.

In another implementation, a computer-implemented method comprises identifying genetic sequencing data for a first patient; identifying one or more genetic variants in the genetic sequencing data; and for particular ones of the identified genetic variants, identifying the patient with respect to contracting a disease that corresponds to the genetic variant. The identifying is based on variant classifications determined by a process that includes: identifying, by a computer server system, stored electronic data that represents genetic sequencing for one or more genes for individuals in a population of patients who have submitted to genetic sequencing; generating, for each of multiple individuals and from the stored electronic data, probability data for the individuals and probability data for relatives of the individuals, the probability data representing likelihoods that a particular person corresponding to the probability data carries a deleterious mutation in a particular gene; and generating a score for a genetic variant, wherein the score is a function of probability data for the individuals and for relatives of the individuals, and the score represent a composite probability that a certain variant is a deleterious or benign variant.

In another implementation, a computer-implemented system is disclosed. The system includes a server sub-system storing data that characterizes genetic sequencing information for a plurality of probands, and medical history information for the plurality of probands and relatives of the probands; and a variant reclassifier implemented using one or more processors arranged to access the data and program information so as to perform operations that include: identifying, by a computer server system, stored electronic data that represents genetic sequencing for one or more genes for individuals in a population of patients who have submitted to genetic sequencing; generating, for each of multiple individuals and from the stored electronic data, probability data for the individuals and probability data for relatives of the individuals, the probability data representing likelihoods that a particular person corresponding to the probability data carries a deleterious mutation in a particular gene; generating a score for a genetic variant, wherein the score is a function of probability data for the individuals and for relatives of the individuals, and the score represent a composite probability that a certain variant is a deleterious or benign variant.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram of a reclassification process.

FIG. 2 is a schematic diagram of a computer system for classifying genetic variants from a population of previously-tested patients.

FIG. 3A is a flow chart of a process for classifying genetic variants.

FIG. 3B is a flow chart of a process for classifying and treating a patient using data from the process described n FIG. 3A.

FIG. 4A shows classification thresholds used by the example processes described herein.

FIG. 4B shows minimum numbers of eligible probands before a variant was considered to be eligible for the analyses described herein.

FIG. 4C shows BRCA1 two-fold cross-validation results using composite variants.

FIG. 4D shows BRCA2 two-fold cross-validation results using composite variants.

FIG. 4E shows BRCA1 and BRCA2 validation results using true variants with previously determined classifications.

FIG. 5 shows an example of a computing device that may be used to implement systems and processes described in this document.

In the drawings, like reference numbers refer to similar elements throughout.

### DETAILED DESCRIPTION

Described herein are systems and techniques for classifying (including by reclassifying) genetic variants that are believed to have a possible causal connection to one or more diseases. The variants may be represented in existing data that was obtained through sequencing (e.g., germline sequencing) of the DNA of a large number of patients (also referred to here as probands or individuals). Such patients may also have provided additional data, including information about cancers or other relevant diseases with which they have been diagnosed, and their age at diagnosis, and corresponding disease/age information for their family members (plus data that indicates the type of relation with each such family member, e.g., sibling, parent, grandparent, aunt/uncle, cousin, etc.). Each such patient's personal and family history may then be analyzed by computer for a list of diseases of relevant concern, such as cancers associated with the BRCA1 and BRCA2 genes. Familial and patient-specific weighting and probability tables may then be constructed that indicate the likelihood that particular variants exhibited by each patient are or are not deleterious, based on empirical analysis of the dataset of patients. Such empirical analysis may reflect the actual rates of the relevant disease (in addition to age of onset or diagnosis, tissue type, etc.) of patients in the relevant dataset (either having or not having a deleterious mutation). More particularly, empirically-observed probabilities of having a deleterious mutation in a certain gene in sets of patients who have the same health condition and the same severity of the family history (i.e., given the patient age and sex, diagnosis, and age at diagnosis, and relative strength of family history of the disease) may be identified using a computer system.

The probability tables may be constructed by identifying others in the patient population who have deleterious variants, and stored as a data structure in a computer database. For family tables, closer relations (e.g., parents and siblings) may be stored and identified, weighted more heavily than further relations (aunts/uncles, and cousins). Also, the patients in the population who are considered for the computer analysis may be limited to patients who were tested around the same time as each other, and around the same time as a patient whose condition is currently being checked, such as all patients tested within n months of the main patient, where n may be 1, 2, 3, 6, or 12-up to a level that will prevent bias from different timing of the tests but still permit a statistically significant number of subjects to be included in the computations.

Scores for each of the relevant variants may then be computed by determining a likelihood ratio for each patient that relates the posterior odds to the prior odds of the patient not carrying a deleterious mutations. Such a computation reflects how much more likely the particular patient is to be mutation-free than mutation-carrying given what the system knows about the diagnosis and family history. This increment may be kept for each patient, (1−p)/p where p is the empirical probability of having a deleterious mutation in the gene in question.

For each variant, the likelihood ratios for each of the patients can be multiplied by each other to determine a score for the corresponding variant using a computer sub-system. Such scores may then be used to indicate whether the particular variant is likely deleterious or not, so as to move a classification from an uncertain level to a more certain level. Such reclassification may be used by a physician or other caregiver in relation to the care provided to a particular patient who has relevant variants in their sequenced and tested DNA. For example, a physician may order more-frequent-than-normal testing for the patient for a particular type of cancer, or may assign the patient regular testing exercises so that the patient can identify the onset of a cancer. Other appropriate treatment approaches may also be used, consistent with standard of care for patients who are understood to be susceptible to a particular disease.

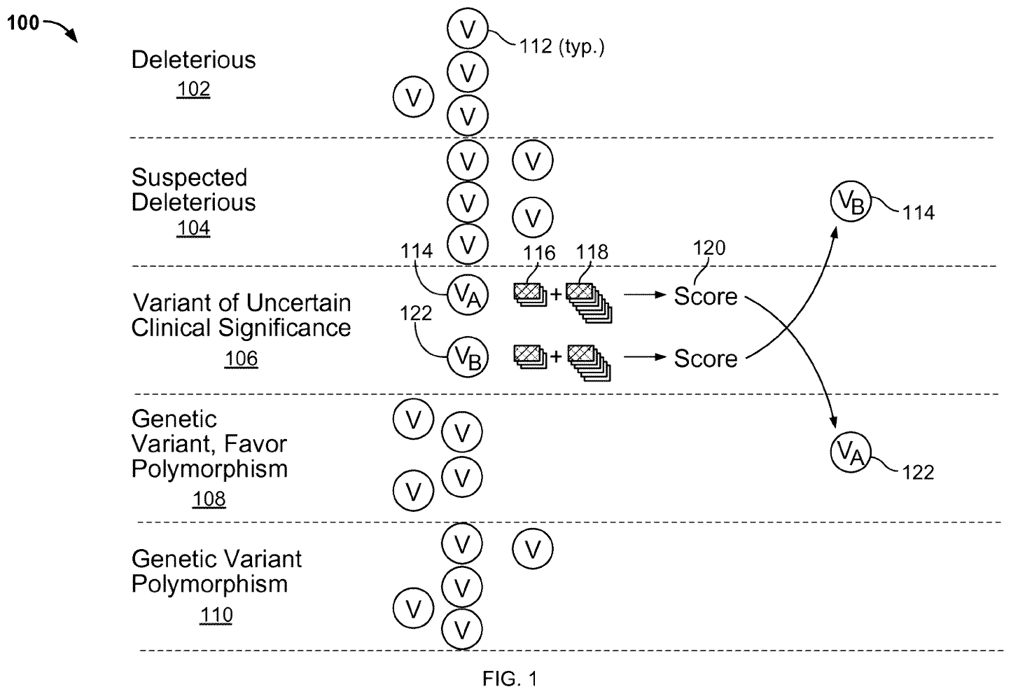

FIG. 1 is a conceptual diagram of a reclassification process. The process follows the general approach to reclassification just discussed. In general, the figure shows a number of variants that have been classified by a system 200 operating on one or more computer sub-systems, into one of five categories, and two of which need further classification because their initial classification was uncertain. The particular classification scheme shown here reflects the scheme for BRCA1 and BRCA2 genes.

Each classification in this representation occupies a horizontal band in the figure, with the name of each classification shown at the left edge of the respective band. The bands, in order from most clinically severe classification to least clinically severe are: deleterious 102, suspected deleterious 104, variant of uncertain clinical significance (VUS) 106, genetic variant, favor polymorphism 108, and genetic variant polymorphism 110. Various circles are shown to represent particular patients, or probands 112, who have a relevant variant, and have been classified according to standard mechanisms into one of the five classifications 102-110. In this example, two of those probands 114 and 122 have a VUS classification 106. Under such a classification, the probands are somewhat in limbo—not knowing whether they are relatively safe or at real risk—which is a poor situation.

As shown by the flow path to the right of probands 114 and 122, however, those probands can be reclassified by a process indicated by the flow path. In particular, the variants exhibited by probands 114 and 122 can be analyzed across a large populations of probands who have been subject to germline level sequencing and surveying, for analysis and weighting of each proband's personal and family disease history (e.g., for different breast cancers). The acquired information includes the proband's age, ethnicity, and personal cancer (or other disease) history (including cancer types and age of, and severity at, diagnosis if applicable), and the proband's history of cancer including a list of affected relatives, cancer type(s) and age(s) at diagnosis, in addition to the relation of each affected relative to the proband.

Proband-specific probability tables 116 for the particular variant can then be constructed for each studied proband (or may have previously been constructed), as can family weighting tables 118, for particular genes that are being studied. Such tables may be constructed on an ongoing basis in a system as probands are added to a database (e.g., as additional probands are tested and surveyed), and the overall statistics in the system may be updated periodically or upon the addition of each new proband. Separate probability tables, or other data representations of probabilities associated with particular variants and particular diseases that correspond or may correspond to those diseases may be constructed independently for different genes, such as for the BRCA1 and BRCA2 genes. The weighting tables may represent, for each family member, the proportion of deleterious mutation carriers in the studied population that has the same cancer type and a similar age at diagnosis. The probability tables may represent, for each proband, the proportion of deleterious mutation carriers in the studied population that has the same cancer type and a similar age at diagnosis.

The probability tables are used in combination to generate a score 120 for each variant. In determining such a score, a likelihood ratio is computed for each proband in the system that relates posterior odds to prior odds of the proband not carrying a mutation that is deleterious. A likelihood ratio for the overall variant may then be computed by cumulatively multiplying the likelihood ratios of each of the probands of the system, where the result may be referred to here as a score for the variant. The weight given to any member of the system may be provided a decreasing weighting in the score based on the member being further away in family relation, such that, for example, data for siblings is weighted more heavily than is data for grandparents of the proband.

As shown in the figure, the score may be used to reclassify the variants 114, 122 into new classifications. For example, the score generated from the probability tables for variant 122 indicates that the variant is more associated with deleterious results than was originally suspected, so the variant 122 is reclassified as a suspected deleterious classification 104. Such reclassification may occur by changing the actual classification of the variant 122 within a series of classifications that are provided by an outside standard, or may occur by leaving the classification in place, but providing additional information for delivery to a healthcare provider so that the provider can identify the patient as needing more intense treatment than someone that who appears with a VUS variant. Such additional information may include, for example, the computed score along with information for interpreting the score (e.g., which could include a URL that links to such information where a lab report is provided electronically).

Thus, by the techniques generally described here and more specifically described below, a particular patient can receive additional information from a diagnosis that is based on genetic sequencing for the patient, including by analyzing probabilities that particular probands and relatives of probands are likely to have mutations that are deleterious, and based on observed occurrences of cancers in a studied population and optionally ages of the patient and probands, and ages at which others in the population were diagnosed with the relevant diseases or diseases (or were determined to have passed a predefined stage of the disease such as by analyzing the date of diagnosis and the severity of the diagnosis when it was made, e.g., a patient diagnosed with a stage IV cancer may be assigned an earlier diagnosis date as compared to a patient diagnosed with stage I cancer).

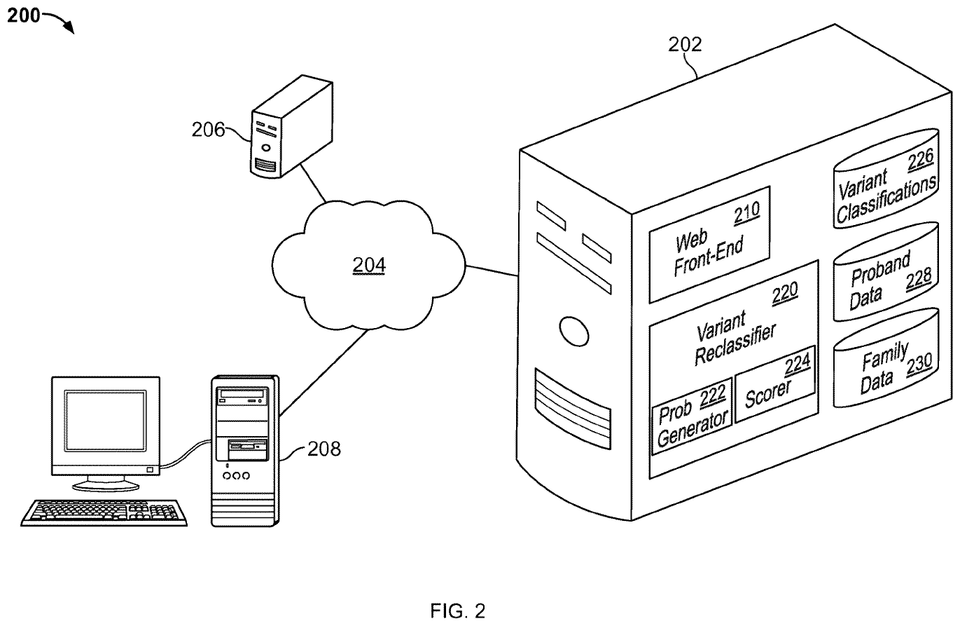

FIG. 2 is a schematic diagram of a computer system 200 for classifying genetic variants from a population of previously-tested patients. In general, the system is configured to receive, essentially continuously, genetic sequencing data about various probands, along with initial or updated medical histories for the probands and their families. The system is programmed to determine, from such information, indicators, such as numeric scores, for particular genetic variants, where the scores represent combinations of likelihoods that probands and family members of probands in the system 200 who have the variant, are to have a deleterious disease or diseases associated with the variant.

Referring now to particular components of the system 200, there is shown a client computer system 208 and a server computer system 202 connected by a network 204, such as the internet. A supplemental server system 206 is also connected through the network 204, such that each of the computer systems 202, 206, and 208 can communicate with and obtain information from each other, While shown in particular forms for purpose of illustration, the various computer systems 202, 206, and 208 can take various appropriate forms. For example, server system 202 may be implemented in a rack server in a data center, including as a virtualized application on a server system whose hardware is shared with other applications and even other organizations (though with appropriate security actions in place).

In this example, the server system 202 includes a web front-end 210 through which various users at computers like client computer system 208 can access the server system 202. The web front-end provides an interface (e.g., receiving HTTP requests and providing code (e.g., HTML, CSS, and JavaScript)) for interacting with a user of client computer system 208, such as through a native application or web browser. Typical operations performed through the web front-end include uploading of information about particular probands, including uploading of electronic files that characterize the genetic sequencing for the proband and family history information that is submitted via an API defined by or for an operator of the server system 202. For example, client computer system 208 can display forms that can be completed by an operator and/or the operator may upload or point to files that are accessible to the client computer system 208, the server system 202, or both. As one example, the server system 206 may store genetic sequencing data for a variety of individuals, and the operator of client computer system 208 may provide to the server system 202 an identifier of the server system 206 along with credential information that allows server system 202 to obtain relevant sequencing data from the server system 206.

In another example, a user at client computer system 208 may upload genetic sequencing information and personal and family history information to the web front-end 210, and may be presented with a classification and other related information for the patient. Such classification may identify variants present in the patient's sequence and may place the patient into a predetermined classification level like those discussed above for FIG. 1. Such classification may follow from or be accompanied by a reclassification of the patient using the techniques discussed here. In certain circumstances, the web front-end may provide information identifying the patient's classification and also information to help a caregiver interpret the classification and other data relating to the patient.

The data presented to a user by the web front-end 210 may be prepared via operations performed on one or more microprocessors implementing a variant reclassifier 220. In general, the reclassifier 220 is programmed to analyze and compute probability and weighting data for probands and for family members of probands (via probability generator 222), where the probability data indicates the probability of a particular person carrying a mutation that is deleterious, based at least in part on observations of other members of the system who have the same or similar mutations and the frequency with which such other members have reported diagnoses of the presence of a particular disease suspected or known to be associated with the mutation. Such probabilities for particular members of the system 202 can be gathered into an overall score for a particular variant via scorer 224, which may be programmed, for example, to consecutively multiply the probabilities of a number of members, such as members identified via genetic sequence analysis as carrying the particular variant. The generated score may be a single representation that represents a number of factors, including likelihoods for probands and for family members, and may be expressed as a number, character, or other alphanumeric representation.

The variant reclassifier 220 may depend on and access data in a variety of data stores in carrying out the programmed operations described herein. For example, proband data 228 is data about particular patients who are entered as members of the system 202. Such data may be stored in a manner that maintains privacy for the patients, such that no personally identifiable information is stored. Rather, when a new patient account is added in the system 202 (e.g., when a technician is uploading new patient data via client computer system 208). As one example, the system 202 can assign each proband an identification number, and the number can be associated with personally identifiable information for the proband only at the organization that submitted the information and is caring for the proband. The particular data for each proband can include genetic sequencing data (e.g., in a predetermined standard file format), gender, age, ethnicity, region, age of diagnosis of certain diseases, weight, and other such information.

Family data 230 includes information for family members of probands. Such information may be linked back to particular probands in the proband data 228 data store, so that likelihoods can be determined for probands based on their and their family history in the manners discussed in this document. The family data generally does not include genetic sequencing data (though it may), and generally includes information about relevant genetically-related diseases for which the relatives have been diagnosed and the ages at which they were diagnosed, their genders, types of relation (e.g., sibling, cousin, parent, etc.), and other relevant information.

The variant classifications 226 stores data about various genetic variants and the relationships between those variants and particular diseases. The variant classifications 226 may also store probability data computed by the variant reclassifier 220 and other similar data needed for determining the correlation between particular variants and particular types of diseases (e.g., types of cancers).

By this system 200 then, a caregiver (e.g., a lab technician) may upload genetic sequencing and medical history (personal and family) for one or more patients the caregiver is providing care to. The caregiver may correlate that data to an account at a system such as system 206 so that the genetic sequencing data for the patient or patients can be accessed by a system such as system 202. The caregiver may then be provided with information that classifies the patient or patients into particular predetermined and accepted classifications, and additionally or as part of the classification, includes information for disambiguating an ambiguous classification-such as by pushing the classification up to a deleterious classification or down toward a benign classification. As a result, the system 200 may provide better diagnostic information and permit a caregiver to treat a patient in much improved manners.

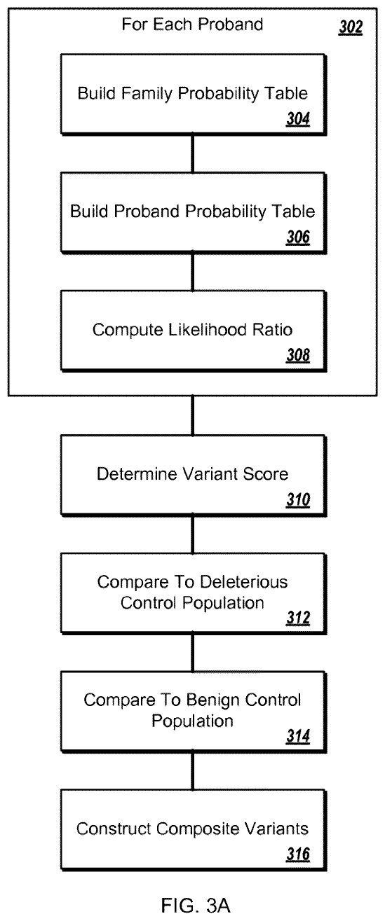

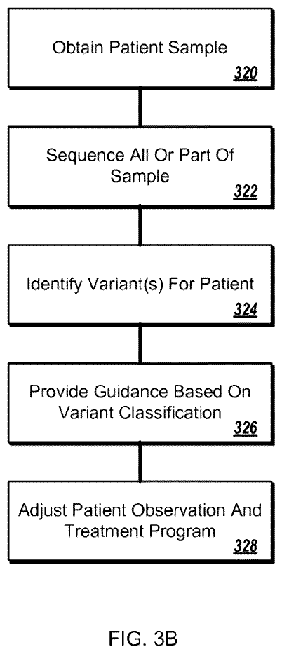

FIG. 3A is a flow chart of a process for classifying genetic variants. Such process may be performed by a system such as system 200 shown in FIG. 2. In general, the process involves building likelihood tables for each proband in a system—both for the proband and for family members of the proband—where the probability tables indicate likelihoods that certain mutations are deleterious, based on the presence of disease diagnoses by other individuals registered in the system who have the mutations. Such probabilities are then used to generate a score for a variant, which score may be checked against controls and may then be used in providing additional diagnostic information to individuals registered with a system that maintains relevant information about a large number (hundreds of thousands or millions) of individuals. The process begins at box 302, which includes steps that are repeated for each proband, or individual patient, registered. Such steps may be performed, for example, as a healthcare provider (e.g., a lab technician or other form of technician) submits genetic sequencing data and medical history data for the proband, and each time such data is updated (e.g., by indicating that particular relatives have recently been diagnosed with a particular cancer or other disease).

Three example sub-processes are shown here for each such proband submission. First, a family weighting table may be built based on the family medical history for the proband. Such medical history may simply indicate genetically-related diseases diagnosed for family members, the age at which the diagnoses were made, and the relation of each such family member to the proband. In more advanced cases, full genetic sequencing data may be available for one or more of the family members (e.g., they may have previously been registered as a proband with the system). As discussed previously, the family probability tables can be constructed independently for each gene under study, and where, for each family member, the probability of a proband carrying a deleterious mutation in the relevant gene is estimated as the proportion (Pᵣₑₗ) of deleterious mutation carriers in the available clinical population that have the same cancer (or other disease) type and similar age at diagnosis. The age range can be set initially and adjusted larger until a statistically acceptable number of probands can be obtained (e.g., 100 probands, with a window as large as +/−5 years). If an adequate number cannot be identified, the age/cancer type probability can be eliminated from the probability table.

At box 306, a proband probability table is built that estimates the probability of the proband carrying a deleterious mutation at each of the genes of interest. The probability represents the proportion (Pₚᵣₒ) of deleterious mutation carriers among members of the population that share the proband's cancer type, age at diagnosis if affected or current age if unaffected, and the sum of Pᵣₑₗ rounded to a nearest integer, with the value of Pᵣₑₗ that is used in the summing being doubled for first degree Family members (i.e., parents, siblings, and children) or otherwise increased relative to the others by a determined level. The weighting for particular family members may also be more finely adjusted, e.g., with first-degree members weighted at 1.0, second-degree at 0.5 (e.g., grandchildren, grandparents, aunts, uncles, nieces, and nephews), and others at lower levels as appropriate.

At box 308, a likelihood ratio is computed for each proband, where the likelihood ratio relates the posterior odds to the prior odds of the proband not carrying a deleterious mutation. If a proband or a relative had multiple diseases, the highest applicable Pᵣₑₗ and/or Pₚᵣₒ value can be used in calculating Pₐᵥₑ, where Pₐᵥₑ is the average Pₚᵣₒ value for all probands (with and without deleterious mutations). In terms of formula, then, the likelihood ratio (LR) is computed as:

((1−Pₚᵣₒ)*Pₐᵥₑ)/(Pₚᵣₒ*(1−Pₐᵥₑ)),

If one assumes independence of individuals with VUS, likelihood ratios of individuals can then be cumulatively multiplied to obtain a likelihood ratio for the variant, which may be referenced here as the score (PS) of the variant (box 310):

PS=(LR₁)(LR₂)(LR₃) . . . (LRₙ).

Such analysis may exclude members of the population for whom personal or family history was not available, or who carried a deleterious mutation or VUS within the relevant genes, or carried additional deleterious mutations or variants (i.e., in addition to the one(s) being analyzed). Comprehensively tested (e.g., sequenced) family members may also be eliminated in certain instances so as to lessen bias. Also, where more than 100 observations (or another determined statistically relevant number) are available, only the most recent 100 probands may be analyzed that otherwise meet the other requirements just mentioned.

At box 312, the result (e.g., in the form of scores) is then compared to a control population, so as to evaluate the significance of each score. At box 314, the data can also be controlled to a benign control population, and at box 316, composite variants are constructed so as to better understand the predictive capabilities of the approach described above.

In one actual example, deleterious controls were selected from individuals known to carry a deleterious sequencing mutation within the relevant gene of interest, and benign controls were selected from individuals with no variants in the relevant genes, or variants known to be benign. A minimum of 100 unique control individuals (or some other number determined to be adequate) were randomly selected from a population for each carrier proband, and were matched to each proband by ethnicity and the date the testing occurred (with a goal of matching the date of testing as closely as practical). If the proband specified one ancestry, all controls were selected from that ancestry, and if it specified two ancestries, controls were selected from each ancestry (at least 50 controls from each). Upon matching of the positive and negative controls to each carrier proband, 100,000 deleterious and 100,000 benign composite variants were constructed for score comparison. Each composite variant was composed of the same number of control probands as the variant that was under investigation. For example, if the investigational variant had been observed in 20 eligible probands, then 100,000 deleterious and 100,000 benign composite variants, each with 20 probands (matched to their respective investigational probands) were constructed. Due to limited control availability, in most cases, the same control was used for the construction of more than one composite variant. The scores were then calculated for each composite variant and plotted on a phenograph to illustrate the deleterious and benign composite variant contributions.

A minimum number of qualifying carrier probands was required before a variant became eligible for analysis. Proband minimums were dependent on gene and variant classification call (“Deleterious” or “Polymorphism”) made by the analysis tool. These minimums were established as follows. Four two-fold cross-validations were performed. In each validation, the proband dataset was divided randomly into two equal halves, consisting of ˜208,000 probands per half. Probability tables (see above) were constructed using half A. Composite variants were constructed from half B probands (and vice-versa). For deleterious composite variants composed of n individuals, n variants were chosen uniformly at random from true deleterious variants within the gene of interest to allow for representation of common and rare deleterious variants. One individual was selected uniformly at random from each chosen variant. These n individuals were treated as carriers of the composite variant to be classified. Each proband after the first proband selected had a 0.05 probability of being randomly duplicated as an additional proband in order to simulate unknown relatedness of two probands carrying the same variant. Composite variants with n probands were constructed for each gene and classification starting with n=1 and increasing in increments of 1. A minimum of 10,000 composite variants was constructed for each n. Polymorphic composite variants were similarly constructed. Positive (PPV) and negative (NPV) predictive values were generated for each n and variant classification combination as follows:

$$\begin{matrix}PPV = P ( truth + I call + ) \\ \sim = \frac{( TP / N + ) \times Prevalence}{( TP / N + ) \times Prevalence + ( FP / N - ) \times ( 1 - Prevalence )} ,\end{matrix}$$

$$\begin{matrix}NPV = P ( truth - I call - ) \\ \sim = \frac{( TN / N - ) \times ( 1 - Prevalence )}{( TN / N - ) \times ( 1 - Prevalence ) + ( FN / N + ) \times Prevalence} ,\end{matrix}$$

where N+ is the total number of tests involving deleterious variants, N− is the total number of tests involving polymorphic variants, TP is true positives, TN is true negatives, FP is false positives, and FN is false negatives.

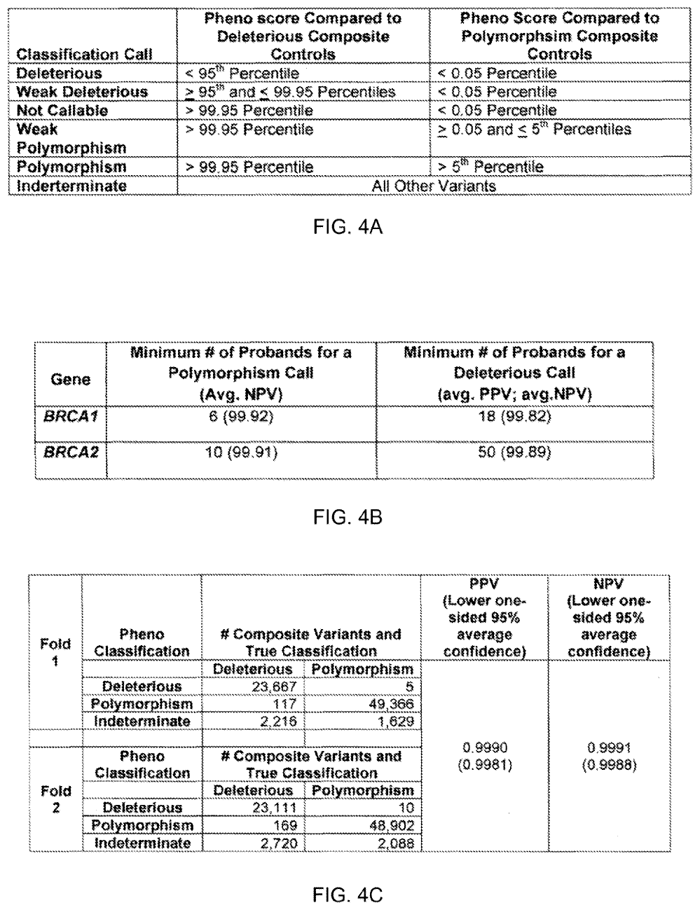

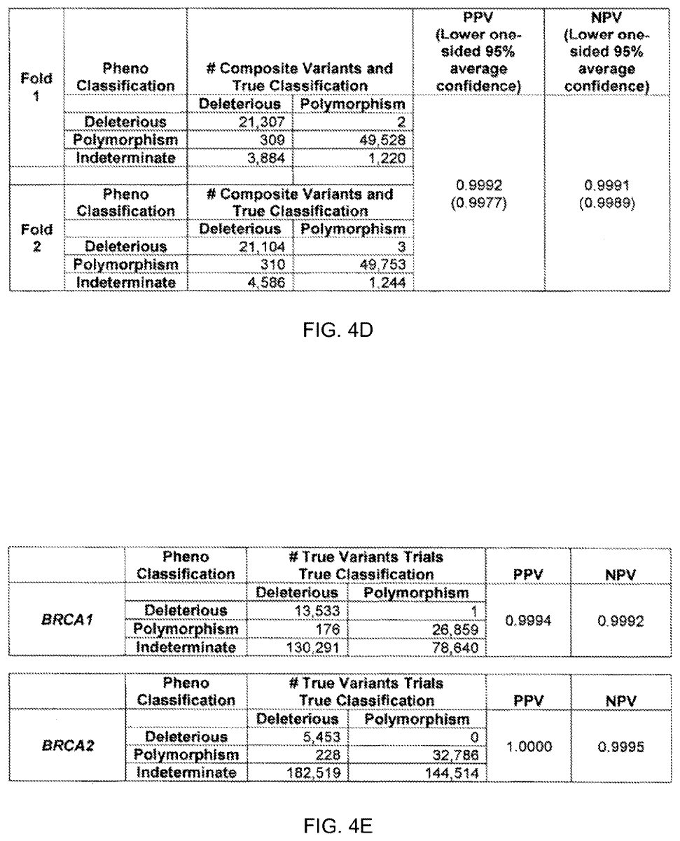

Deleterious mutation prevalence estimates of 13.8% and 6.7% were used for BRCA1 and BRCA2, respectively. These prevalence values are based upon the estimated representation of deleterious mutations within the VUS populations for each gene. Thresholds for classification calls were predefined, with only “Deleterious” and ‘Polymorphism” calls being counted toward PPV and NPV (see FIG. 4A). All other classification calls were categorized as “Indeterminate” for the purpose of determining proband minimums. Minimum proband numbers resulting in PPVs and NPVs of >0.995 by gene and classification were selected for further validation and implementation of the analysis tool (see FIG. 4B).

A two-fold cross-validation was performed utilizing composite variants. For each gene, the proband dataset was divided into two halves as defined above. Scores were generated for a minimum of 50,000 composite polymorphisms and 25,000 composite deleterious mutations per gene. Each variant was initially analyzed using the minimum number of eligible probands required (FIG. 4B). If the tool was unable to call a variant either as “Deleterious” or “Polymorphism,” the number of probands was increased by at least 20% more than the initial proband minimum number, and the score was recalculated. This process was repeated until either the variant was classified as “Deleterious,” “Polymorphism,” or “Not Callable.” PPVs and NPVs were calculated for each gene.

Validation with true variants was also performed, via actual BRCA1 and BRCA2 variants previously classified using other methodologies. For each variant tested, the minimum number of probands was selected uniformly at random and a call made, If the variant could be classified as “Polymorphism” or “Deleterious,” the call was scored. Otherwise, the number of probands was increased by an additional 20% and another call attempted. Probands were repeatedly added in this manner until either a definitive call was made or no more carriers were available. 100 trials were performed for each variant analyzed, and PPV and NPV calculated.

Analysis of BRCA1 and BRCA2 variants was performed by constructing BRCA1 and BRCA2 probability tables, as described above using an entire proband set of ˜416,000 individuals. For variants with less than 100 probands, all eligible probands were used for analysis. Probands were excluded from analysis if personal and family history was not provided, they carried a deleterious mutation (sequencing or large rearrangement) or VUS in addition to the variant under analysis, or were a relative of another carrier proband. For variants with more than 100 eligible probands, only the most recent 100 probands were analyzed. Proband minimums, established through development and validation, were utilized.

The approach described here was validated and developed based on a dataset consisting of ˜416,000 probands undergoing BRCA1 and BRCA2 sequencing analysis with or without large rearrangement analysis. The approach was validated against both synthetic composite BRCA1 and BRCA2 variants and true variants identified within an actual patient population. PPV and NPV were calculated to assess validity. Two-fold cross-validation with a minimum of 50,000 composite polymorphisms and 25,000 composite deleterious mutations per-gene demonstrated that both the PPV and NPV for the BRCA1 and BRCA2 genes were >=0.999 (scc FIGS. 4C and 4D). Validation using true variants with clinical classifications previously determined by other methodologies also demonstrated PPV and NPV>=0.999 (see FIG. 4E). The analyses of true variants were based on 100 trials of each eligible variant.

The approach described here was also used to analyze three BRCA1 variants, c.181T>G (p,Cys61Gly; Deleterious), c.5096G>A (p.Arg1699Gln; Suspected Deleterious with reduced penetrance), and c.1065G>A (p.Lys355Lys; Polymorphism), and three BRCA2 variants, c.2808_2811 del (p.Ala938Profs*21; Deleterious), c.7878G>C (p.Trp2626Cys; Suspected Deleterious with reduced penetrance), and c.7242A>T (p.Ser2414Ser; Polymorphism), which had been previously classified based on other methodologies. The approach correctly classified p.Cys61Gly and P.Ala.938profs*21 as “Deleterious” and p.Lys355Lys and p.Ser2414Ser as “Polymorphisms.” p.Arg1699Gln and p.Trp2626Cys, which most likely represent hypomorphic alleles based on segregation and/or functional analyses, were classified as “Not Callable” by the approach, consistent with their previous hypomorphic classifications.

FIG. 3B is a flow chart of a process for classifying and treating a patient using data from the process described n FIG. 3A. In general, the process involves receiving data for a particular proband, or patient, and using information generated by the techniques discussed above to classify the particular proband's variant(s) and/or provide additional information to assist in diagnosis and treatment of the patient.

The process begins at box 320, where a patient sample is obtained. Such a sample may be obtained at various physical locations and various points in time. For example, the sample may be provided to a clinic and held for analysis at the clinic or mailed to another center for analysis. The patient may also provide the sample at home (e.g., via swab) and mail it to an analysis center. The particular technique for obtaining the sample, as long as it is accurate, is not critical. With the sample, the patient may also provide personal (e.g., age, gender, and ethnicity) and medical history information, both for the patient and family members of the patient, as discussed in more detail above. Such history data may be entered into a computer system using a predefined electronic form, and may be stored in a database of information, including in a manner so that no personally identifiable information about the patient may be obtained.

At box 322, all or part of the sample is genetically sequenced, in various conventional manners so as to obtain a digital representation of the genetic sequence. Such data may be stored in a file according to an applicable standard, and can be transmitted from the location where it is captured to other locations (e.g., from server system 206 to server system 202 in FIG. 2). The file that includes the electronic sequence data may be accessible to a common computer system with a file or files storing the personal and medical information provided by or on behalf of the patient.

At box 324, variants for the patient are identified. Such identification may occur by using the computer system to compare the patient's sequenced data to known variants so as to identify which of those known variants the patient exhibits. The variants may then be analyzed according to classification decisions made for such variants using techniques like those discussed above, in terms of classifying and reclassifying the variants, such as by generated scores for the particular identified variants. Also, other scores may be generated that show that the patient's data should be interpreted in a particular manner. Such additional data may be analyzed and provided for the patient only upon determining whether the patient would otherwise be classified according to a VUS or other ambiguous classification (e.g., for which the patient is not treated in an intensive manner but is also not classified in a manner that would indicate that the patient is likely free from disease risk).

At box 326, guidance is provided based on the variant classification. For example, the patient may be reclassified into a different standard classification group than they would have without the additional analysis. Alternative, the caregiver who submitted the information or another caregiver for the patient may be provided with an indication of what the reclassification analysis identified, along with information to help the recipient better interpret the data. For example, the person may be provided, via electronic communication over a network (e.g., the Internet) from the computer system, a reclassification score for the patient and one or more sentences, tables, or other things that indicate what that means (e.g., that indicate what percentile the patient is within a particular group). The information may be more detailed, including showing detailed data that led to the reclassification (rather than just a simple score) and additional written information about how the reclassification is computed and what it means (e.g., in an electronic communication, in a document attached to an electronic communication, and/or on a web page or other internet-accessible document that is pointed to by a URL provided to the requester).

At box 328, an observation and treatment program for the patient is or is not adjustment based on the provided guidance. For example, a patient may be told additional information in helping the patient determine whether a mastectomy or other elective operation is advised. Alternatively, or in addition, the patient may be placed into a program of more intense observation (e.g., more frequent than usual checkups) and the reclassification may be used to obtain insurance coverage for such a program, or the patient may pay separately for the additional interaction. The patient may also be scheduled for additional tests, such as imaging, biopsies, and other tests that would normally be available to a patient who was initially classified at a higher level for the particular disease. Other similar steps may also be taken, in the discretion of the relevant physician, to better care for the patient in view of the additional information that is available as a result of the reclassification process.

FIGS. 4A-4E depict tables that report on results on the testing of the classification/reclassification techniques discussed above with respect to BRCA1 and BRCA2 data for a large number (˜416,000) of probands in a population. FIG. 4A shows classification thresholds used by the example processes described herein. FIG. 4B shows minimum numbers of eligible probands before a variant was considered to be eligible for the analyses described herein. FIG. 4C shows BRCA1 two-fold cross-validation results using composite variants. FIG. 4D shows BRCA2 two-fold cross-validation results using composite variants. FIG. 4E shows BRCA1 and BRCA2 validation results using true variants with previously determined classifications.

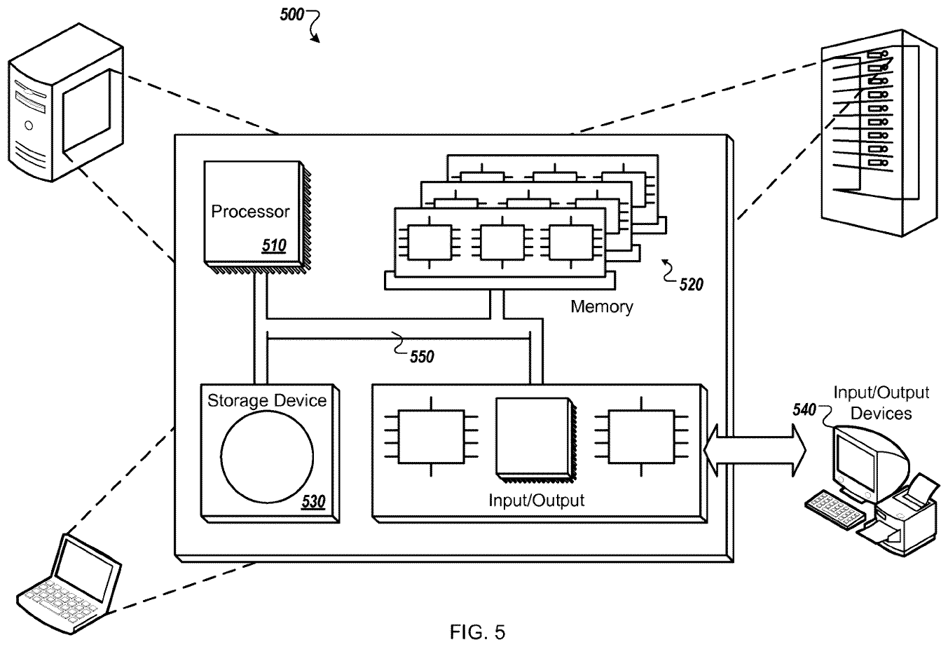

FIG. 5 is a block diagram of computing a device 500 that may be used to implement the systems and processes described in this document, as either a client or as a server or plurality of servers. The system 500 can be used for the operations described in association with any of the computer-implement methods described previously, according to one implementation. The system 500 is intended to include various forms of digital computers, such as laptops, desktops, workstations, personal digital assistants, servers, blade servers, mainframes, and other appropriate computers. The system 600 can also include mobile devices, such as personal digital assistants, cellular telephones, smartphones, and other similar computing devices. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 640 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor. “Machine-readable medium” is therefore distinguished from “computer-readable medium.”

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback), and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms for performing the systems and processes described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A system for variant reclassification using probability tables, comprising:
one or more processors coupled to non-transitory memory, the one or more processors configured to:
maintain a genetic sequencing data for a set of individuals, the genetic sequencing data comprising a variant of a breast cancer (BRCA) gene for each individual of the set of individuals;
generate a probability table based on genetic information selected from the set of individuals that have a respective variant of the BRCA gene, the probability table comprising probability data for the set of individuals representing a likelihood that a particular individual carries a respective mutation in the BRCA gene;
receive, via a web-based frontend, first genetic data of a first individual not included in the set of individuals;
determine that the first genetic data classifies the first individual as having a variant of uncertain clinical significance (VUS) with respect to the BRCA gene;
responsive to determining the first genetic data indicates the first individual as having the VUS classification, reclassify the first individual based on the probability table and the first genetic data; and
provide, via the web-based frontend, the reclassification of the first individual.

2. The system of claim 1, wherein the one or more processors are further configured to:
reclassify the variant further based on genetic data of a relative of the first individual indicating a deleterious variant of the BRCA gene.

3. The system of claim 1, wherein the one or more processors are further configured to:
generate the probability table by identifying frequencies with which those of the set of individuals are diagnosed with a pathogenic outcome of a disease associated with the BRCA gene.

4. The system of claim 1, wherein the one or more processors are further configured to:
reclassify the first individual by generating a score for the first genetic data, the score generated based on the probability table.

5. The system of claim 4, wherein the one or more processors are further configured to:
reclassify the first individual as having a deleterious variant of the BRCA gene upon the score satisfying a threshold.

6. The system of claim 1, wherein the one or more processors are further configured to:
generate the probability table further based on family history data for each individual in the set of individuals.

7. The system of claim 1, wherein the one or more processors are further configured to:
receive the first genetic data of the first individual in one or more electronic sequence files corresponding to the first individual.

8. The system of claim 1, wherein the one or more processors are further configured to:
update a treatment plan for the first individual to include one or more tests or procedures.

9. The system of claim 8, wherein the one or more tests or procedures comprise one or more of an imaging procedure, a biopsy, or a physical examination.

10. The system of claim 1, wherein the BRCA gene comprises a BRCA1 gene, a BRCA2 gene, or a combination thereof.

11. A method of variant reclassification using probability tables, comprising:
maintaining, by one or more processors coupled to non-transitory memory, a genetic sequencing data for a set of individuals, the genetic sequencing data comprising a variant of a breast cancer (BRCA) gene for each individual of the set of individuals;
generating, by the one or more processors, a probability table based on genetic information selected from the set of individuals that have a respective variant of the BRCA gene, the probability table comprising probability data for the set of individuals representing a likelihood that a particular individual carries a respective mutation in the BRCA gene;
receiving, by the one or more processors, via a web-based frontend, first genetic data of a first individual not included in the set of individuals;
determining, by the one or more processors, that the first genetic data classifies the first individual as having a variant of uncertain clinical significance (VUS) with respect to the BRCA gene;
responsive to determining the first genetic data indicates the first individual as having the VUS classification, reclassifying, by the one or more processors, the first individual based on the probability table and the first genetic data; and
provide, via the web-based frontend, the reclassification of the first individual.

12. The method of claim 11, further comprising:
reclassifying, by the one or more processors, the variant further based on genetic data of a relative of the first individual indicating a deleterious variant of the BRCA gene.

13. The method of claim 11, further comprising:
generating, by the one or more processors, the probability table by identifying frequencies with which those of the set of individuals are diagnosed with a pathogenic outcome of a disease associated with the BRCA gene.

14. The method of claim 11, further comprising:
reclassifying, by the one or more processors, the first individual by generating a score for the first genetic data, the score generated based on the probability table.

15. The method of claim 14, further comprising:
reclassifying, by the one or more processors, the first individual as having a deleterious variant of the BRCA gene upon the score satisfying a threshold.

16. The method of claim 11, further comprising:
generating, by the one or more processors, the probability table further based on family history data for each individual in the set of individuals.

17. The method of claim 11, further comprising:
receiving, by the one or more processors, the first genetic data of the first individual in one or more electronic sequence files corresponding to the first individual.

18. The method of claim 11, further comprising:
updating, by the one or more processors, a treatment plan for the first individual to include one or more tests or procedures.

19. The method of claim 18, wherein the one or more tests or procedures comprise one or more of an imaging procedure, a biopsy, or a physical examination.

20. The method of claim 11, wherein the BRCA gene comprises a BRCA1 gene, a BRCA2 gene, or a combination thereof.
